(19) United States
(12) Patent Application Publication
(10) Pub. No.: US 2014/0155753 A1
(43) Pub. Date: Jun. 5, 2014

(54) DISPOSABLE LIGHT SOURCE FOR ENHANCED VISUALIZATION OF SUBCUTANEOUS STRUCTURES

(71) Applicants: James E. McGuire, JR., Westerville, OH (US); David M. Callard, Palo Alto, CA (US); Robert L. Crane, Dayton, OH (US); Richard D. Peters, Gahanna, OH (US); Thomas L/ Harris, Powell, OH (US); Steven H. Mersch, Germantown, OH (US)

(72) Inventors: James E. McGuire, JR., Westerville, OH (US); David M. Callard, Palo Alto, CA (US); Robert L. Crane, Dayton, OH (US); Richard D. Peters, Gahanna, OH (US); Thomas L/ Harris, Powell, OH (US); Steven H. Mersch, Germantown, OH (US)

(21) Appl. No.: 14/236,384

(22) PCT Filed: Aug. 1, 2012
(86) PCT No.: PCT/US2012/049231

§ 371 Date: Jan. 31, 2014

Related U.S. Application Data
(60) Provisional application No. 61/513,689, filed on Aug. 1, 2011.

(51) Int. Cl. A61B 5/00

(52) U.S. Cl. CPC A61B 5/0082; A61B 5/0075; A61B 5/489
USPC 600/473; 600/476

(57) ABSTRACT

A disposable light source device for the non-invasive visualization of veins, arteries or other subcutaneous structures of and objects in the body, or for facilitating and monitoring intravenous insertion or extraction of fluids, including a conforming layer for interfacing and optically coupling with the body surface, and adhering the device to the body portion, and a main light source for directing near infrared light through the conforming layer to illuminate the body. The disposable light source device can also include a light transmissive and electrically insulative layer that is disposed between and electrically insulates the main light source from the body-contacting conforming layer. The disposable light source device can also include a proximity sensor that controls activation of the first light source such that the light source is on only when the conforming layer is brought into proximity to the body surface.

### CROSS-REFERENCE TO RELATED APPLICATIONS

This application is a US National Stage of International Application No. PCT/US2012/049231, filed Aug. 1, 2012 (pending), which claims the benefit of US provisional application 61/513,689, filed Aug. 1, 2011 (expired), the disclosures of which are incorporated herein by reference in their entirety.

### BACKGROUND OF THE INVENTION

The present invention relates generally to medical devices and procedures, and more particularly to a disposable light source structure useful in conjunction with systems and methods for enhancing visualization of veins, arteries and other subcutaneous structures of the body in the administration of medical treatment to a patient and in the diagnosis and management of disease.

The administration of medical care of a patient often requires vascular access, including in emergency situations. It is well settled that expeditious administration of medical care to the victim improves the prospects of recovery for the victim. Patients may have veins that are partially collapsed, or veins that are difficult to find or difficult to access (such as in the treatment of infants or geriatric persons), which further complicates procedures for gaining access to the veins. The treatment of patients requiring vascular access may also be complicated by patient size, obesity, skin pigmentation or other physical characteristic.

US Patent Publication US 2007-0032721 (the disclosure of which is incorporated herein by reference) discloses a multi-layered structure in the form of a disposable patch useful in conjunction with procedures for the non-invasive visualization of veins, arteries or other subcutaneous structures of the body or for facilitating intravenous insertion or extraction of fluids, medication or the like. The patch is particularly useful in conjunction with systems and methods for the detection and display of subcutaneous structures such as described in U.S. Pat. No. 6,230,046 to Crane et al (the disclosure of which is incorporated herein by reference), which describes systems and methods for enhancing the visualization of veins, arteries or other subcutaneous natural or foreign structures in the body and for facilitating intravenous insertion or extraction of fluids, medications or the like in the administration of medical treatment to a patient, including a light source of selected wavelength(s) for illuminating or transilluminating a selected portion of the body and a low-level light detector and suitable filters for generating an image of the illuminated body portion.

US Patent Publication US 2004-0215081 (the disclosure of which is incorporated herein by reference) discloses a real-time visualization and detection of an extravasated or infiltrated fluid in subdermal or intradermal tissues at a site of an intravascular injection by illuminating an intended site of an intravascular injection with infrared light from a light source and generating real-time images of the body and the injected fluid to determine differences in contrast evidencing extravasation or infiltration of said fluid near the vasculature within the body.

It is known that while light emitted in the near infrared and infrared spectrum is not viewable by the unaided human eye, sufficiently high and/or prolonged emissions of infrared light into the eye of a human could result in eye injury and damage such that care should be taken to avoid the emission of infrared light, including near infrared light, to patients, medical staff or bystanders whose eyes are unprotected.

In the practice of the procedures for visualization of subcutaneous structures by trans-illumination using either visible, infrared or near infrared light, proper support of the light source in order to accurately direct the light onto a body portion of interest may be an awkward or inefficient procedure for the health care provider in treating a patient. There remains the need for improvements in the features and functions of a hands-free, light source device for directing light from the light source onto and through the body portion of interest in the imaging process.

### SUMMARY OF THE INVENTION

The present invention relates generally to medical devices and procedures, and more particularly to a single use or disposable light source (DLS) device that includes a light-directing and transmitting structure that can be applied to the skin surface of a portion of the body and a light source supported by the structure, including, but not necessarily limited to, an infrared light source. The device provides illumination of a body portion, and is useful in conjunction with systems and methods for real-time non-invasive visualization and identification of veins, arteries and other subcutaneous structures and objects in the body, in the administration of medical treatment to a patient, including facilitating intravenous insertion or extraction of fluids, medication or the like, and various surgical and diagnostic procedures affecting veins and arteries. The illumination can include transillumination, side illumination and backscattering. It can also include reflection by holding the light source away from the body and detecting light that is reflected back from the patient. In addition, this light source permits the detection and identification of other natural subcutaneous structures and foreign objects such as metallic object or other non-natural items that could be present as a result of an accident or placed in situ for prosthetic purposes.

The invention relates to a disposable light source (DLS) device for use in medical imaging procedures in the visualization of subcutaneous structures of a body portion, comprising an optically transparent conforming layer having a first surface configured for interfacing in intimate laminar contact with the surface of a body portion of interest to adhere the DLS device to the body portion and to provide optical coupling with the body portion, the conforming layer having a second surface opposite the first surface, and a first light source for emitting light through the conforming layer and illuminating the body portion of interest, and further including electrical circuitry adapted for selectively activating the light source whereby the body portion of interest is selectively illuminated through the conforming layer.

The invention can further relate to a DLS that includes a proximity sensor for detecting when the DLS is positioned in proximity to the surface of the body portion of the patient. The proximity sensor controls the flow of current to the light source, and turns on the light source only when the light-emission pathway of the DLS is in close proximity to or in contact with the body portion, and which can turn off the flow of current of the light source when the DLS is removed from proximity to the body portion. The proximity sensor significantly limits and preferably prevents light, especially near infrared and infrared light, of the DLS from emitting generally in a direction other than the body portion, to avoid inadvertent light emissions that would become noise in the detected image or could enter the eyes of the patient, medical staff, or bystanders.

The invention can further relate to a DLS that includes an activation mechanism that prevents the flow of current to the light source until activation. The activation mechanism can be an on-off button, or a switch including a toggle, sliding or rocker switch. The activation mechanism can be a removable breaker, such as a non-conductive removable tab that is disposed between the battery and the circuit to block the flow of current until removed.

The invention further relates to a DLS that utilizes electrical power for the light source, and that includes a layer or film of an electrically insulating material as a means for isolating electrically the light source, and an optional proximity sensor, from the body portion of the patient. The layer or layers of electrically insulating film or coating material prevents any electrical current flowing from or to the light source and associated electrical components of the DLS from flowing through the potentially electrically conductive conforming layer that is in direct contact with the skin of the body, thus avoiding and preventing electrical shocks or sensations or from interfering with additional medically placed instrumentation or sensors in the vicinity of the light source. In addition, the isolating layer also insulates the body surface from the heat generated by the near infrared light emitting diodes commonly used for illumination purposes associated with imaging the internal structures of the body.

The invention can further relate to a DLS that includes a light source wherein the source of electrical power and a controller for the light source are disposed remote from the DLS, to minimize the components, features, cost and complexity of the DLS. The simplicity of the design and components of the attachable and disposable light source can significantly reduce the cost of such device, allowing its use in a wider variety of medical procedures involving vascular access and subcutaneous imaging of the vasculature and the structures, or objects (endogenous or exogenous) with the body.

The invention can further relate to a DLS that includes a disposable or replaceable light source, and a reusable structure that holds and electrically connects the light source and proximity sensor to a source of power and control.

The invention can further relate to a DLS wherein the power source can be disposed as a battery on the DLS or PCB thereof, or from a remote device.

In another aspect of the invention, the light source in the DLS includes a light emitting diode (LED) emitting at a selected or selectable wavelength.

An aspect of the invention is a DLS having a plurality of spaced-apart light sources (typical LEDs), in an array including two, three, four, five, six, or more light sources that improves the diffusion of light through the body portion, and improves the imaging of the body portion.

In another aspect of the invention, the DLS includes and supports the light source for use in a hands free manner.

In a further aspect of the invention, the DLS is adhesively attachable to a body portion of a patient. In yet a further aspect of the invention, the DLS can be adhesively attached to a body portion of the patient in close conformance to the topography of complex surface features of the skin of the body. In yet another aspect of the invention, the DLS can be gently attached to the body and peeled off after use without causing discomfort or damage to sensitive skin, such as that of neonates, pediatrics and the elderly. The present invention is also useful to illuminate the tissue of burn patients who do not yet have a new layer of dermis. For example, the device of the invention can be used to assess the extent of burn damage for which current methods are painful. Hydrogel, when used as the conforming layer, can act as a secondary skin and its adhesion is good enough to be used on raw muscle with minor support. Later the extent of new skin formation can be assessed for its viability after grafting, which requires a very gentle attachment of the light source, and the hydrogel is sufficient for this purpose if additional contact pressure is maintained.

In another aspect of the invention, the DLS includes the use of a battery and secondary nIR absorbing films used to control the light output instead of a controller for use in primitive settings where electrical power and space for a control system is unavailable, e.g. battlefield or accident scenes in remote settings.

In another aspect of the invention, the DLS facilitates sterile administration of a medical procedure affecting vasculature or other subcutaneous structures using sterile covers designed to fit the light source or by sterilizing the light source itself and providing it as a sterile component.

In a further aspect of the invention, the DLS couples optically with the surface of the body portion of a patient in illuminating the body portion with light of selected wavelength.

In another aspect of the invention, the DLS has a light source that pulses under the control of a controller, including an associated image receiving, processing or detecting device, in order to dissipate heat generated when the light source is on and prevent discomfort or burning of the patient, and/or to maximize the signal to noise ratio by increasing the peak nIR emission when pulsed and thereby increasing significantly the ratio of meaningful light to interfering light.

In another aspect of the invention, the DLS deters or prevents disease transmission from patient to patient since it is disposed of after use on a patient.

In another aspect, the DLS provides a manipulable and mobile light source(s) during medical procedures when attached to or associated with a separate, manipulable implement, to facilitate the positioning or placement of the DLS near or against the skin of the patient, or near, on or within the body of the patient.

Further improvements in the DLS described herein provide a device that is simpler to operate, that minimizes the disposal of parts and elements, and that is compatible with illumination detecting devices and visible, infrared and near infrared image processing and viewing apparatus.

These and other aspects of the invention will become apparent as a detailed description of representative embodiments thereof proceeds.

### BRIEF DESCRIPTION OF THE DRAWINGS

The invention will be more clearly understood from the following detailed description of representative embodiments thereof in conjunction with the accompanying drawing figures wherein,

FIG. 1A shows a perspective view of an embodiment of a disposable light source device of the invention.

FIG. 1B shows electronic components of the disposable light source.

FIG. 2 shows an exploded view of the structural materials of the disposable light source.

FIG. 3 shows an axial section view of the disposable light source of FIG. 1, displaced a distance from a body portion to be transilluminated.

FIG. 4A shows the disposable light source of FIG. 3, positioned on the body portion to be transilluminated.

FIG. 4B shows another embodiment of the disposable light source having two light sources, positioned on the body portion to be transilluminated.

FIG. 5 shows a perspective view of another embodiment of a disposable light source device of the invention including a reusable component and a disposable component.

FIG. 6 shows the structure of the disposable light source device of FIG. 5.

FIG. 7 shows the reverse view of the reusable component and disposable component of the disposable light source device of FIG. 6.

FIG. 8 shows an exploded view of the structure of the disposable component portion of the disposable light source device viewed in FIG. 7.

FIG. 9 shows a sectional view of another embodiment of the disposable light source device.

FIG. 10 shows an exploded view of another embodiment of the disposable light source device.

FIG. 11 shows a schematic of an exemplary circuit on a printed circuit board of the disposable light source device.

FIG. 12 shows a typical LED drive schematic useful in the disposable light source device of the invention.

FIG. 13 shows a sectional view of another embodiment of the disposable light source device.

FIGS. 14A and 14B show a DLS and a mounting handle for the DLS, and the DLS mounted to the handle for manipulation by the user.

FIG. 15 shows a schematic of a circuit of an optical proximity sensor of the disposable light source device.

FIG. 16 shows a schematic of a circuit of a capacitance proximity sensor of the disposable light source device.

### DETAILED DESCRIPTION OF THE INVENTION

The present invention provides a disposable light source (DLS) for illuminating, from outside the body, subcutaneous structures of and objects within the body, so that the resulting reflection or transillumination of such subcutaneous structures of or objects within the body can be detected, enhanced and visualized externally of the body portion. The emitted illumination from the light source passes through the conforming (e.g., hydrogel) layer structure and through the body-contacting surface of the conforming layer. With the DLS placed on the selected body portion, and with the body-contacting surface of the hydrogel structure (conforming layer) in intimate contact with the skin or other surface of the body portion, the emitted illumination passes through the skin and into and through of the body portion. In order to minimize the reflection of the emitted illumination at the skin interface and to efficiently illuminate the subcutaneous body structure or object, the refractive index of hydrogel (conforming layer) should be closely matched to the refractive index of the body portion of interest.

Near infrared (nIR) light can be used to visualize veins and arteries for, among other things, the introduction of intravenous catheters and needles into patients when used with an appropriate image conversion detector, as described in Crane et al. (U.S. Pat. No. 6,230,046), the disclosure of which is incorporated by reference in its entirety. The image conversion detector may be any real time imaging instrument such as night vision goggles or imaging sensors based upon a number of solid state technologies such as charge couple devices (CCD), complementary metal oxide semiconductor (CMOS) detector, hole accumulation device (HAD) or similar solid state or image tube based imaging producing electronic device. Other devices may be used, such as micro-bolometers, vacuum photodiodes, photomultipliers, photoconductors devices both intrinsic and extrinsic, photovoltaic devices, pyroelectric detectors, or other devices capable of producing real time images using niR and infrared light.

The necessary illuminating light is introduced to the patient (human or animal) with either a single or an array of nIR light emitting diodes (LED, and the resultant image of the illuminating and transilluminating light passing through the body is observed with one or more image producing devices. LEDs can be energized with a simple direct current power source such as a battery, from an associated image conversion detector, or commercial mains. The LEDs can also be powered with au alternating current power source that has been rectified and filtered to provide direct current at the appropriate voltage necessary to provide the light output for imaging, It is also possible to use other light generating devices, including organic light-emitting diodes (OLEDs) and xenon sources used for other medical imaging as in sigmoidoscopy or even laser sources of light. Broad band light emitting sources may also be used for this purpose such as thermal sources, although they are much less efficient as only a small fraction of their output is used to generate an image, Furthermore chemical reactions may also be used for this purpose such as chemiluminescent or bioluminescent sources (e.g., light sticks).

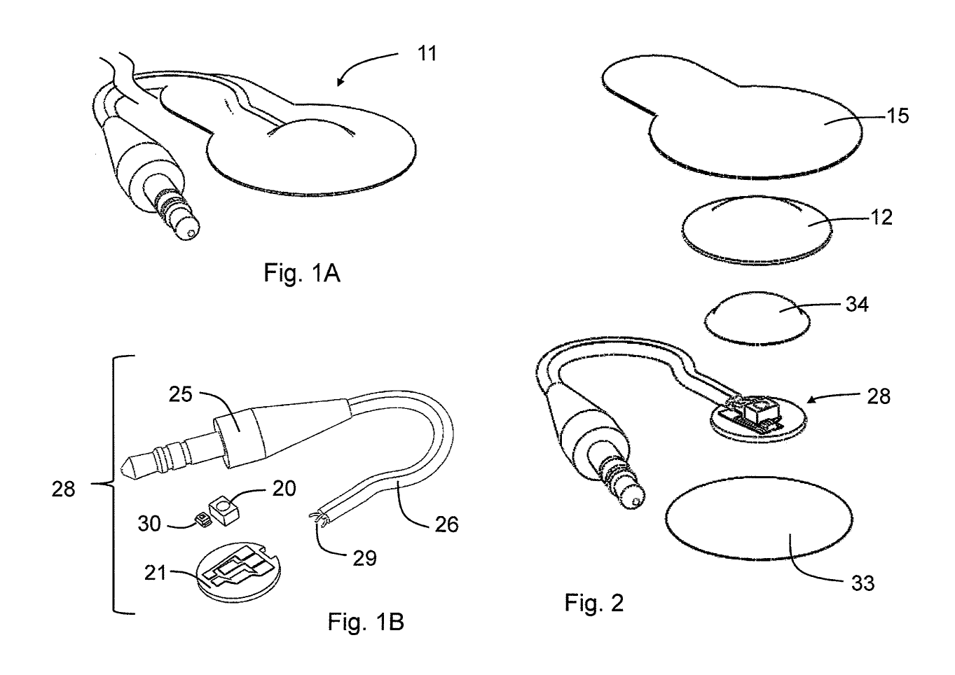

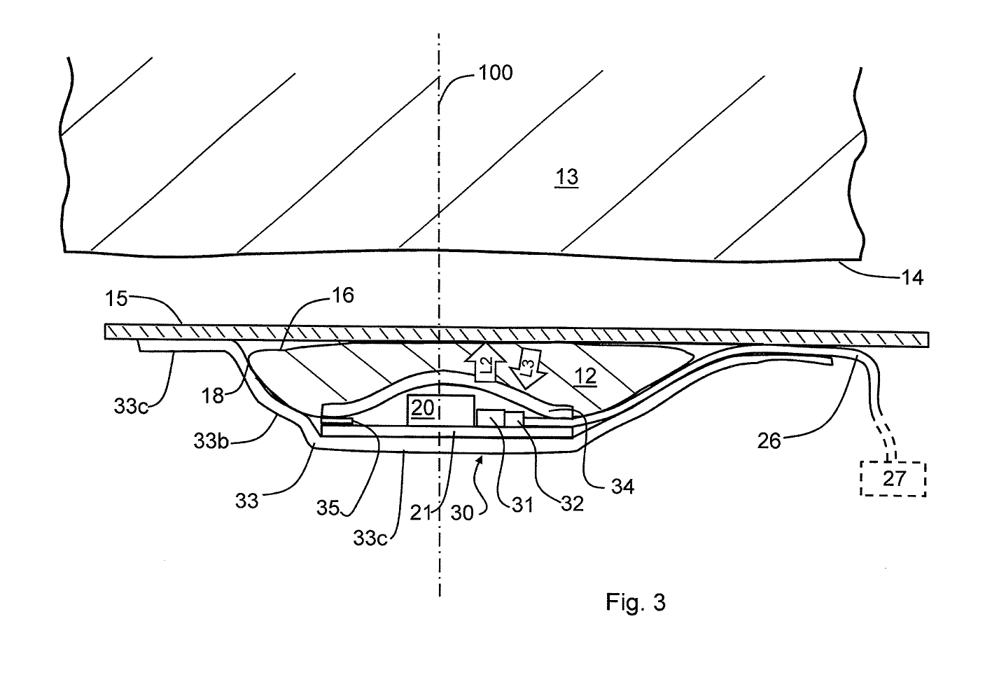

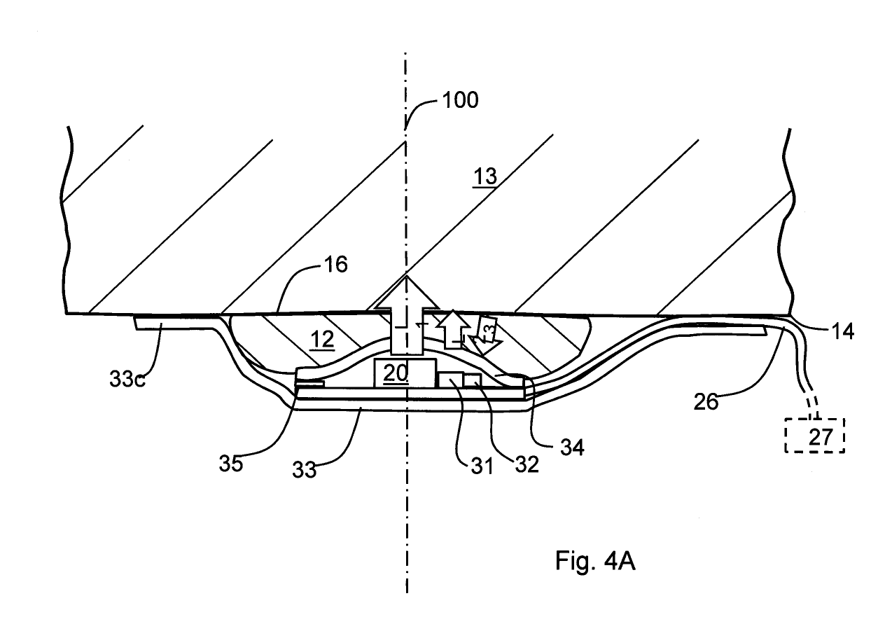

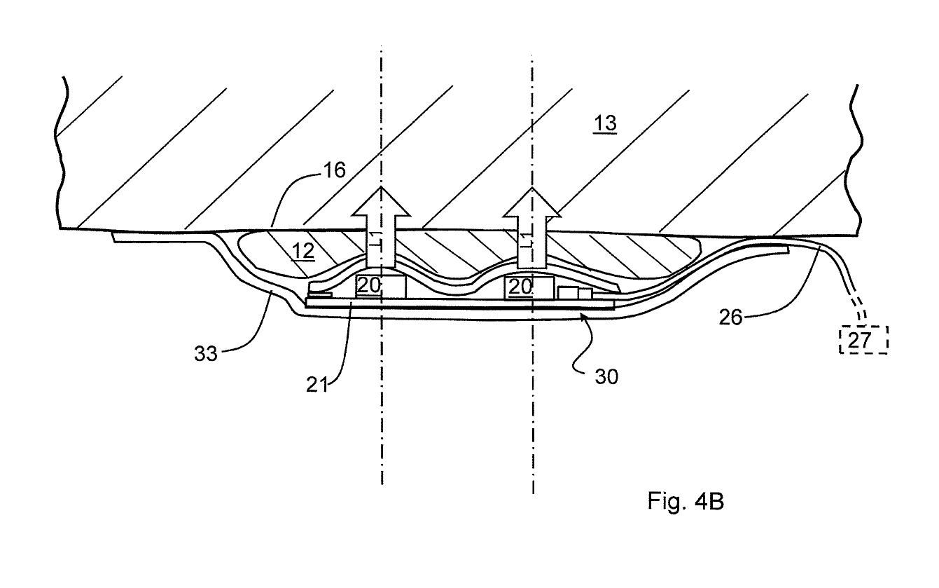

FIG. 1A shows a DLS 11 structure representative of the invention along with the supporting structural elements that render the structure a complete unit. DLS 11 can be configured to facilitate the illumination of a body portion of a patient with visible (V), infrared (IR) or near infrared (nIR) light for visualizing the vasculature or other subcutaneous structures of and objects within the body. FIGS. 1B and 2 show the electronic components of the DLS and the structural material and films of the DLS, respectively. FIGS. 3 and 4A show cross sectional views of the DLS 11 at a position away from the body portion, and at a position affixed to the body portion, respectively.

FIG. 1B shows the electronic components 28 of the DLS that include a light source 20 for emitting light for visualizing a body portion, a circuit board 21 for mounting the light source 20 and associated circuitry and connection of the light source 20 with a proximity sensor 30. The DLS 11 is connected to an electrical connector 25 with wiring 26 and connections 29 to the circuit board 21. The electrical connector 25 is a standard plug for connection with external power and control devices, as discussed hereinafter.

FIG. 2 shows an exploded view of the structural material and films of the DLS associated with the electrical components 28, including a conforming layer 12, a reflective layer or film 33, an optional electrically insulative or non-conductive material 34, and an optional barrier release layer 15.

A first substantially optically transparent conforming layer 12 is configured to interface with and provide a layer conforming to the surface 14 of the body portion 13 of interest to be illuminated, as shown in FIG. 4A. The conforming layer, typically consisting of a hydrogel, provides adhesive attachment of the DLS device to a body portion of a patient. Typically the adhesive attachment of the DLS device to a body portion is a self-adhering, hands-free attachment, sufficient to allow the DLS device to remain in position on the body portion during the imaging procedure, without requiring holding or adjustment by hand of the operator. In yet a further aspect of the invention, the DLS can be adhesively attached to a body portion of the patient in close conformance to the topography of complex surface features of the skin of the body. The area of intimate contact between the conforming layer 12 and the body portion is sufficiently large, relative to the weight of the DLS device, to effect and maintain hands-free adhesive attachment.

A protective contaminant barrier release layer 15 of an nIR transparent polymer or other suitable material covers the body-contacting surface 16 of conforming layer 12 for containment and protection of conforming layer 12 and is removed and discarded prior to applying the DLS 11 to the body portion 13 surface 14 of a patient for transillumination.

The light source 20 (such as one or more near infrared (nIR) light emitting diodes (LED), an incandescent source, a chemiluminescent source or other suitable source) may be supported on a non-conductive substrate having electrical conductive circuits or traces. The substrate can include a printed circuit board (PCB) and a flexible substrate. A flexible substrate includes a polymeric sheet that can be bent or rolled without breaking, to improve the conforming of the light-emitting face of the DLS to the skin. An example of a flexible substrate is a polymeric sheet, made of polyimide material or the like, as described in U.S. Pat. No 7,210,817, the disclosure of which is incorporated by reference in its entirety. A flexible printed circuitry laminate can include a composite of metal conductors and dielectric substrates bonded together by an adhesive system. Another flexible substrate can include a copper foil that is electrolytically deposited or rolled annealed to the polymer sheet and does use adhesive.

Light source 20 may comprise a source of light of substantially any wavelength, such as up to about 1.4 microns, and may further comprise any type of light source as would occur to the skilled artisan practicing the invention. Further, source 20 may comprise multiple sources such as two or more LEDs disposed on PCB 21 in any desired configuration, as illustrated in FIG. 4B. For example, a plurality of LEDs can include two, three, four, five, six, or more LEDs and can be arranged in a straight line, a cross, a diamond pattern, a rectilinear pattern, a circular pattern, and an oval pattern. An array of a plurality of light sources can improve the diffusion of light through the body portion, and improves the evenness of the light emitted from the array, which improves imaging of the body portion.

An important issue in the use of trans-illumination for imaging of body portions with nIR light is the wide range of light intensities that need to be transmitted by a device through different human body extremity types and conditions. For example, neonate's and children's hands are relatively thin, and will allow passage of a higher amount of light transmission than, for example, the forearm of an adult male, which is much thicker. It is estimated that the difference in transmission between various body portion types is up to five orders of magnitude (100,000 times). To provide effective imaging across such a wide variation in light intensity, the image processor employs a logarithmic response to light intensity and 16 bit gray scale resolution.

In the practice of visualization methods for which the invention finds wide utility, light source 20 preferably emits light in the nIR in the range of about 0.70 to about 1.1 microns, and, in a specific embodiment of the invention, the light source 20 comprises an LED emitting at about 0.850 microns (using such as OSRAM SFH 4650). The LED light source 20 is oriented on the circuit board (e.g., PCB) to direct emitted light along an axis 100 of the device, substantially transverse to the body-contacting surface 16 of conforming layer 12. The output of an LED can be within a range of about 0.1 mW to 10 W, and typically at least from about 1 mW, or at least about 5 mW, or at least about 10 mW, and up to about 1000 mW, or up to about 500 mW, or up to about 100 mW, or up to about 50 mW. The LEDs are typically driven by a current ranging from about 1 mA to over 1 Amp, depending on the size of the LED. Mid-LEDs are typically driven with between about 20 mA to about 100 mA, with large LEDs driven with between hundreds of mA to more than 1 A.

A means for controlling the current or power delivered by a power source into an LED light source can include adjustment of the resistance between the power source and the LED, which reduces the current flow to, and therefore the radiant flux from, the LED light source. The resistance adjustment can be designed into the circuit board of the DLS, or into a circuit of a remote electrical power and controller apparatus. Alternatively, an adaptor can be provided that includes an inlet port to accept the DLS connector, an outlet plug to connect to the remote electrical power and controller apparatus, and a resistor element to reduce or step-down the current delivered from the remote power and controller apparatus to the DLS. The resistive element can be adjusted manually or from the electrical power and controller apparatus, to increase or decrease resistance continuously or incrementally, to control the current or power delivered by a power source into an LED light source.

The DLS of the present invention can include a plurality of light sources wherein the wavelengths of emission are different as between individual light sources. In particular, the plurality of light sources can include a plurality of LEDs, wherein an LED can emit in a spectrum of wavelengths different from at least one of the other LEDs. For example, one LED can emit nIR light in a range centered on about 850 nm, and a second LED can emit nIR light in a range centered on about 660 nm, while a third LED can emit in a range centered on about 780 nm.

The LED package can include a lens for shaping the emitted light, including a lens to focus and shape the light into a narrow beam, or alternatively to shape the light into a wider beam, or into split beams.

In the illustrated embodiment, the control circuitry and power source for the light source 20 are disposed remote of PCB 21 and from the DLS 11, as within system 27, which can comprise an image acquisition, processing and display system, and may be connected during use to light source 20 by wiring 26 and connector 25. Wiring 26 may comprise two or more electrically conducting wires as needed to provide power and control to the components on PCB 21 and to collect signals from those components, depending on which of the components are disposed on PCB 21 or are remote of DLS 11.

In another embodiment, the LED light source can be a reverse mounted LED. A light-emitting diode (LED) chip or die of the reverse mount variety, or “reverse mounted LED”, is known in the art. Examples of off-the-shelf reverse mount LEDs include but are not limited to HSMx-C4AO LED manufactured and sold by Agilent Technologies, Inc.; SML811 series light emitting diodes by Rohm; and reverse mount L-193 series by LEDopto. The light-generating LED can be mounted in a cup with electrodes electrically connected and molding material (forming an optical dome or acting as encapsulating material) covering the light emitting diode. The LED can be a package LED that is initially manufactured as one device having a heat sink attached to the reflector cup of light source, thereby making LED package a single manufactured device.

The reverse mounted LED or array of LEDs can include any suitable interconnect technology to provide an electrical circuit among the LEDs, the substrate, the power supply, and any control device. Flexible or traditional wiring, solder attachment, conductive pieces, and/or pressure connectors can be used.

To limit the risk of excessive heat build that might result in damage of the skin of the patient, a thermocouple can be in and around light sources or on a skin-contacting surface to monitor the local temperature of the light source and surrounding surface. The epoxy cast may be formed into a shape, such as a dome, and used for directing light from the LED light source. Such optical domes are optically designed based on a software simulation to design the best shape for directing, magnifying, spreading, or otherwise managing the light emitted from the LED and/or the reflector cup, and can include a round shape dome, or any other shape, such as rectangular, triangular, cylindrical, or oval.

As shown in FIG. 3, a thin reflective layer or film 33 extends laterally from the light source(s) 30, with the reflective surface 36 directed in the same direction as the illumination by the light source 30. The layer 33 can include an aluminum, aluminized or metalized polymeric film such as Mylar®, Kapton®, Lexan ® or Teflon® films, or other suitable material, See for example, Spacecraft Thermal Control Handbook: Fundamental Technologies, 2^{nd} Edition, David G. Gilmore, The Aerospace Corporation, American Institute of Aeronautics, 2002. or Gossamer Spacecraft: Membrane and Inflatable Structures Technology for Space Applications (Progress in Astronautics and Aeronautics), C. M. Jenkins, American Institute of Aeronautics, 2001). The layer 33 has a central portion 33a that overlies the PCB 21, and an intermediate annular portion 33b that overlies the periphery 18 of the conforming layer 12, and an outer annular portion 33c that extends beyond the conforming layer 12 for contact with the skin surface 14. The portion of the reflective layer 33c extends a distance from the outer periphery of the conforming layer 12 that is sufficient to maintain adhesive attachment to the skin around the periphery of the DLS assembly, in order to inhibit and prevent emitted light from spilling beyond the side edges of the conforming layer 12 and outside the confines of the DLS package. Light spillage can interfere with the quality of the nIR imaging. Typically the annular portion 33c extends up to 25 mm from the edge of the conforming layer 12, more typically up to 15 mm, or up to 12 mm, or up to 10 mm, or up to 5 mm, and extends at least 2 mm from the edge of the conforming layer 12, or more typically at least 5 mm, or at least 10 mm, or at least 12 mm, or at least 15 mm,

In addition it is possible to use a structured polymeric based film that reflects light due to its structure. An example of such a film is 3M Vikuiti® Enhance Specular Reflective Film, or Radiant Light Film®, or Diamond Grade™, or Scotchlite®, or Entrofilm 893 (available from Entroech Inc., Columbus, Ohio) or other appropriate reflecting film material. The specific film used is chosen for this application based upon its spectral reflection properties near the emission peak of the nIR LED, its conformability, strength, insulating properties, and compatibility with adjacent materials.

The reflective surface 36 of the layer 33 has an adhesive film that substantially matches the adhesive characteristics of the conforming layer 12 to skin 13, so that the device 11 peels easily and in one assembly from the skin 13 when removed. The material used to adhere the DLS to the patient's skin is based upon the same chemistry as pressure sensitive adhesives (PSA) currently qualified for contact with human skin. The adhesive can include one of two main classes of PSAs used in adhesive tape manufacture, which are based on either on natural rubber (cis-polyisoprene) or an acrylic copolymers, commonly either butyl or 2-ethylhexyl acrylate and acrylic acid. While both compositions are capable of providing an adequate adhesive for use on human patients, since a small portion of the population is allergic to natural rubbers, the acrylic copolymer adhesive composition is preferred. The adhesive should permit sufficient adhesion to the patient's skin so that the DLS does not change position during a medical procedure. During this process the electrical lead connecting the DLS to the controller will in general be dangling from their attachment point and exert a peal force on the DLS. The adhesion forces of the DLS patch must not be so great that removal of the patch also removes weakly held skin or epidermis. The balance is between these two conditions, i.e., holding the DLS patch to the skin of the patient, yet permitting easy removal.

There are many possible geometrical configurations for the distribution of the adhesive on the DLS from uniform to textured. The particular arrangement depends on the application and the ease of removal and the procedure to be performed. For example, if the procedure is to be a short term one, then the pattern and extent and specific distribution of adhesive would be limited, say as a series of dots. If the procedure is to be longer, say on the order of a few days, then the adhesive will be thicker and greater in extent. Of course all of this depends on the needs of the procedure and the skin condition of the patient.

The DLS structure in any of the embodiments described herein according to the teachings of the invention may be of any convenient size consistent with the size of the person on which the DLS is used, and thicknesses of the individual layers may be selected by the skilled artisan practicing the invention within the intended scope of these teachings, the thicknesses hereinabove recited as included in demonstration of the invention being representative only. It is noted further that the invention may find use in veterinary medicine as well as for human application. The overall size of the DLS is about 0.25 to 6 inches in diameter and about 0.10 to 0.5 inch in overall thickness for use on an adult person or a neonate of pediatric patient. Minimum size of the DLS may be limited by the size of the light source selected for use in conjunction with the DLS and by the size of the body features of infant patients.

i.) proximity sensor

The DLS device of the invention can, but does not have to, include a proximity sensing device, illustrated as a proximity sensor, to control activation of the main light source. Activation of the light source, or emitting of light at high power levels, can be limited to only when the DLS is in intimate proximity or direct contact with the surface of body portion of interest.

In a first embodiment illustrated in FIG. 3, the proximity sensor 30 includes a light emitter shown as LED 31 and a photodetector 32, which are connected electronically to the circuitry of the PCB 21. Optionally, the photo detector can include an integral nIR bandpass filter adjacent to the DLS light source. Light L2 can be emitted from the proximity sensor LED 31 when the device is connected to power. Light L3 reflects off the surface 14 of a body portion 13 because of the Fresnel mismatch in refractive indices between air and skin.

In one aspect of the invention, the proximity sensor circuitry detects the lag time between the out-going pulse of light L2 and the returned pulse of light L3. When the body-contacting surface 16 of the DLS 11 is a distance (for example, inches to feet) from the surface 14 of the body portion 13, as shown in FIG. 3, the lag time will exceed a predetermined time interval, and power will be withheld from the main light source 20 by the controller 27. However, when the body-contacting surface 16 of the DLS 11 is a very short distance (for example, millimeters) or in direct contact with the surface 14 of the body portion 13, as shown in FIG. 4A, the lag time will be within a predetermined time interval, and power will be delivered to the main light source 20 by the controller 27, to activate the light source, and light L1 is emitted. When and only when the photodetector 32, or if there are multiple photodetectors, a majority of the photodetectors, receives reflected light L3 back from the LED 31 within the predetermined lag time interval, and optionally within a predetermined power level relative to the emitted light L2, the remote circuitry 27 signals the main light source 20 to activate. The main light source 20 is at all other times deactivated in order to prevent any possible deleterious effects of nIR light on the human body.

In an alternative embodiment of the optical proximity sensor, proximity is determined based on the intensity of the radiant flux of the nIR received by the photo detector in comparison with the light output of the LED light source, as described in more detail hereinafter. In yet another aspect, the photo detector and circuitry inserts a light emission pattern into the proximity light emitter signal, and detects for a corresponding light emission pattern within the reflected light pattern detected by the photo detector. The matching or correlating light pattern can include a distinct light emission pattern that distinguishes the light emitted by the proximity light emitter from other light emissions in the environment or vicinity of the procedure, as described in more detail hereinafter.

Deactivating the main light source 20 when it is not in proximity to the body also prevents any potential damage to the detector, intensifier tube or other optical sensor associated with the image acquisition and processing system used in the visualization of subcutaneous structures utilizing the DLS 11. There can be of course reflections from other components, including the main light source 20 once the DLS 11 is positioned on the body portion 13, so several precautions may be necessary to permit accurate detection of the presence of the skin in close proximity to the light source 20. Firstly, the proximity sensor LED 31 can emit light L2 at a wavelength that is not used by the main light source 20, so these two near infrared light sources, light source 20 and proximity emitter 31, do not interfere functionally with each other. For example, the main light source LED 20 can operate at 850 nm or 0.850 microns, and the proximity light emitter LED 31 can operate at longer wavelengths, usually close to 950 nm or 0.950 microns. Secondly, the pulses of light L2 from the proximity light emitter LED 31 can be timed to avoid the pulses of light L1 from the main light source LED 20 so that there is no overlap of the emissions and thus little in the way of confusion as to the origin of the near infrared light from the main light source 20 and that from the proximity light emitter 31. Thirdly, there can be a number, usually 3 to 4, proximity sensors 30, including proximity light emitters 31, to prevent the possibility that a piece of paper or bed sheet might cover one sensor/proximity light emitter, and thus fool the controller into “detecting” that a body extremity was covering the main light source 20 and thus powering on the main light source 20 prematurely. The number of proximity sensors 30 incorporated into a DLS device depends upon its size, e.g., an adult-sized DLS is larger and may need more LED proximity emitters 31 as compared to a pediatric-sized DLS that typically only uses one LED proximity emitter 31. Thus, it is possible that 3 to 4 proximity emitters may be needed for an adult-sized DLS and only one proximity emitter may be needed for a pediatric-sized DLS. An example of an optical proximity sensor is a Miniature Surface-Mount Proximity Sensor, model HSDL-9100, available from Agilent.

In a typical embodiment, a nIR LED proximity light emitter 31 includes gallium arsenide with a peak emission wavelength in the range of about 0.890 microns to 1.00 microns, such as at about 0.940 micron, and has a silicon photodetector 32 with a peak sensitivity at about 0.9 micron (see e.g., OSRAM reflective interrupter SFH 9201).

In another embodiment of the invention, the proximity sensor can employ the light source 20 itself as the proximity light emitter. The proximity sensor thus can consist of the nIR light source 20 and a photo detector. The photo detector can include a photo transistor or any photo sensitive device, and control circuitry. The photo detector is sensitive to at least one of visible light, ultraviolet light, and infrared light. In a typical embodiment, the photo detector is sensitive to all wavelengths of light, including visible light, ultraviolet light, and infrared light, including the near infrared range. The power and control circuitry is configured to drive the LED light source at a high power-emitting state, for imaging purposes, and at a low power-emitting state, for proximity sensing. The photo detector and circuitry employs the LED light source as a proximity emitter by inserting a light emission pattern into the emitted LED light signal, and matching or correlating the light emission pattern with a light pattern detected by the photo detector. The matching or correlating light pattern can include a distinct light emission pattern that distinguishes the light emitted by the LED light source from other light emissions in the environment or vicinity of the procedure. One example of a distinct light emission is repeated series of one or more short “off” pulses in the emitting light of the LED light source. The controller and circuitry that drives the LED light source to emit the distinct light emission also looks for the same distinct light emission in the light signal received by the photo detector. Receipt by the photo detector of the same distinct light emission then presumed as evidence that the DLS is position proximate (very near) or directly on the surface of the skin, which then changes the LED light source from the low power-emitting state to the high power-emitting state for imaging.

As mentioned above, the power and control circuitry is configured to drive the LED light source at a high power-emitting state, for imaging purposes, and at a low power-emitting state, for proximity sensing. The low power-emitting state is the default state when the DLS device is first powered on. The power and control circuitry employs a low-power threshold of radiant flux of nIR light detected by the photo detector, for powering up the device from low power state to high power state, and a high-power threshold of radiant flux of nIR light detected by the photo detector, for powering down the device from the high power state to the low power state. Typically, the high-power threshold values are based on the predetermined or selected light source power (or radiant flux) output from the light source that is required for the imaging procedure. Electronic and optical-feedback hysteresis is used to prevent the LED from oscillation between high and low power. In a typical use of the device, the DLS operates at the low power state with the photo detector sensing an amount of radiant flux that is below the low-power threshold value. The low-power threshold value is selected or set based on an amount of reflected nIR light (radiant flux) that the photo detector would receive when the DLS is emitting on low power, when placed a first distance (d1) from the skin surface. When the DLS is positioned proximate to or onto the skin surface (a distance closer than d1), the reflected nIR light (radiant flux) received by the photo detector exceeds the low-power threshold, and the DLS is powered up to the high power state, which emits radiant flux in a higher amount suitable for imaging.

In high power operation, the power and control circuitry now compares the amount of radiant flux received by the photo detector during high power operation, to the high-power threshold value. The high-power threshold value is selected or set based on an amount of reflected nIR light that the photo detector would receive when the DLS is emitting on high power, when placed a second distance (d2) from the skin surface, which second distance d2 is typically greater than the first distance d1. Thus, when the DLS operating in the high power state is removed from contact with the skin and removed a distance d2 from the skin, the amount of reflected nIR light that the photo detector receives is less than the high-power threshold value, and the DLS is powered back down to the low power “proximity-sensing” state.

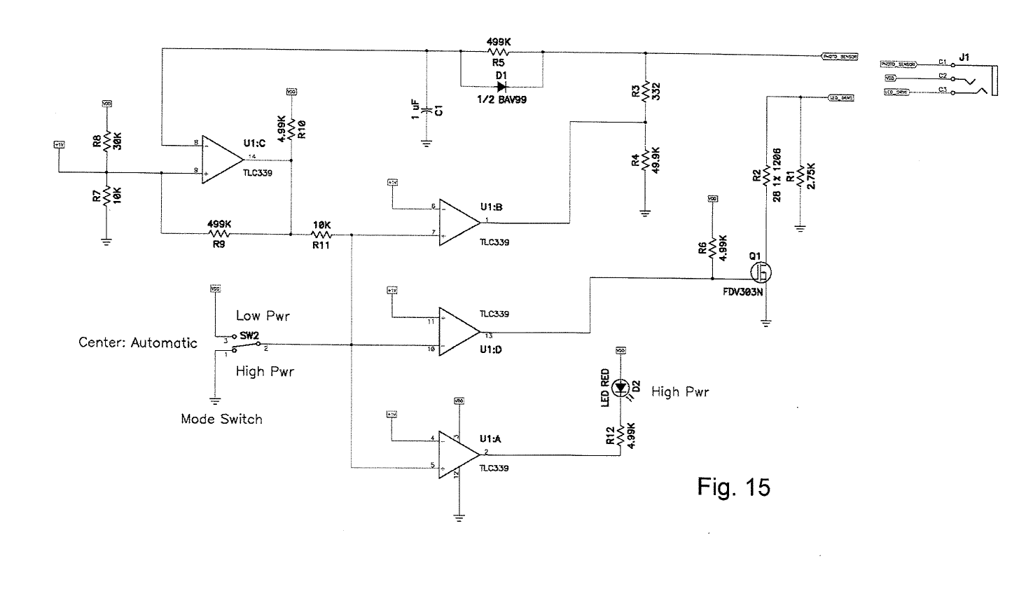

FIG. 15 shows a schematic of an exemplary circuit of an optical proximity sensor of the disposable light source device.

The controller and circuitry can modulate or vary the light emission pattern to a new distinct emission pattern, to prevent a false proximity determination based upon a modulated ambient light source signal with the same distinct light emission pattern.

In addition, potential thermal sensitivity and/or damage to the illuminated body portion 13 is also minimized by controlling the activation of light source 20 only during temporal intervals of image acquisition. In an embodiment of the invention, light source 20 is pulsed (i.e., alternately activated and de-activated) at any selected rate such as at about the flicker fusion frequency of the human eye (e.g., at or above about 30 per second and a 50% duty cycle). Pulsing the main light source 20 allows high light output onto body portion 13 with corresponding lower heat input than if main light source 20 is constantly activated. At a pulse rate above the flicker fusion rate, main light source 20 would nevertheless appear constant. The power drive for the LED can be capacitive coupled to prevent the LED from turning on at full brightness in case there is a failure in the logic pulse drive.

Other arrangements providing the proximity sensing function as would occur to the skilled artisan practicing the invention may be substituted for the arrangement just described without departing from the spirit and scope of the present invention. Examples of other proximity sensing and control methods include capacitive proximity sensor as described in US Patent publication 2009-0216182, or an optical proximity sensor, a mechanical proximity sensor, or an electromechanical proximity sensor, as described in US Patent publication 2008-0091188, the disclosures of which are incorporated by reference in their entirety.

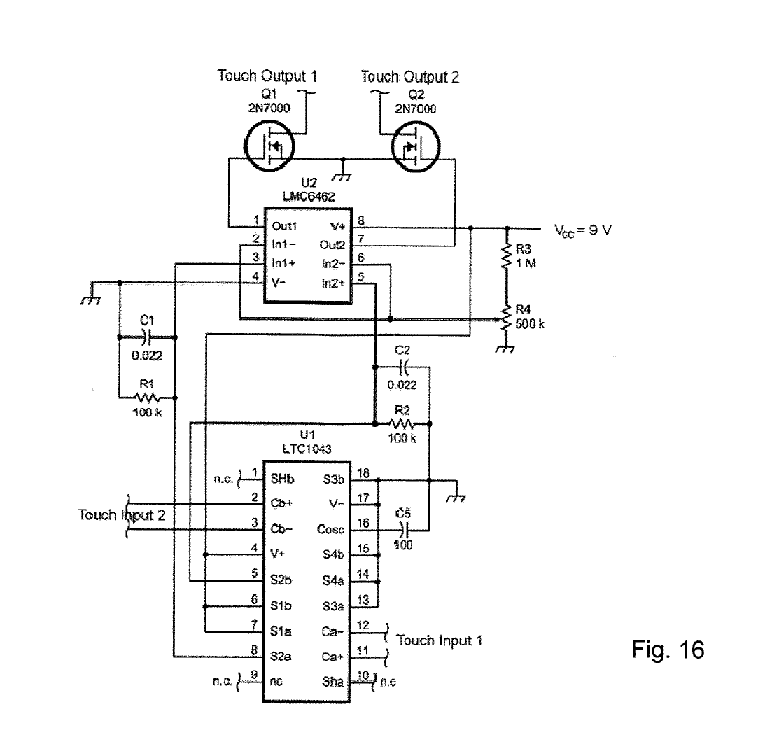

An embodiment of a DLS can include a capacitive proximity sensor. This feature of the DLS allows the controller to sense when the DLS is in touch contact with the human body as a source of capacitive energy. In an embodiment of a capacitive proximity sensor, the printed circuit board (for example, shown in FIG. 3, labeled 21, can have a back surface (not shown, opposite the light source) comprising a layer of solid copper, an antennae for capacitive sensing. The copper layer is electrically connected to at least one conductor in the wiring 26, which then connects with an interface board on the remote electrical power and a controller apparatus 27 via plug 25. The interface board has conventional capacitive sensing circuitry to detect and measure capacitance at the antennae. Another embodiment of a capacitive proximity sensor is illustrated in FIG. 16. This circuit needs a power source that is in the range between 6 and 18 volts for proper operation of a capacitor circuit. A suitable capacitor circuit is the Linear Technology LTC 1043, which is monolithic, charge-balanced, dual switched capacitor instrumentation building block, having a pair of switches alternately connects an external capacitor to an input voltage and then connects the charged capacitor across an output port. The internal switches have a break-before-make action. Typically, a supply of 9 volts is used. Two operational amplifiers (for example, Texas Instruments LMC 6462 or Texas Instruments LM193) increase the signal and match the input impedance needed for the field effect transistors (FET) (2N7000 or equivalent). These four active elements provide a signal that can be used to drive other circuitry to detect the touch of a human hand and thus guarantee that the DLS is in contact with the skin. The devices have two inputs, which can allow an extra margin of safety by placing the DLS in contact with both the patient and an operator. A charge transfer touch sensor that detects one signal rather than the two in the LTC1043 can also be used (for example, Quantum Research QProx QT113). A variable resistor R4 can be used to set the sensitivity of the touch sensor so that either a light touch or a more forceful touch is needed for activation. The value for R4 can be determined via testing in a clinical setting.

ii) electrically insulative layer

The invention further relates to a DLS that utilizes electrical power for the light source, and that includes a layer or film of an electrically insulating material as a means for isolating electrically at least the light source from the body portion of the patient.

As shown in FIG. 3-4A, PCB 21 and any or all of the LED light source 20, any proximity sensor 30 if used, and the distal end 29 of wiring 26 are isolated electrically with one or more layers 34 of a light transmissive, electrically insulative or non-conductive material, including a glass, a plastic, an elastomer or other suitable material selected by the skilled artisan. The electrically insulative layer 34 electrically insulates the light source 20, PCB 21 and other components mounted thereon, and the distal end 29 of wiring 26 to prevent electrical current from flowing into conforming layer 12. This is important when the conforming layer 12 comprises an electrically conductive material, such as hydrogel. The light transmissive, electrically insulative layer 34 may also provide heat insulation around PCB 21. The electrically insulative layer 34 can be resilient or flexible as needed, and may be transparent or opaque to visible light. Examples of such an electrically insulative layer material might be chosen from the one of more of the following material classes a semi rigid thermoset or thermoplastic polymer such as an acrylic polymer poly(methyl methacrylate) a transparent polyester such as polyethylene terephthalate polycarbonate or polybutyrate; or a more flexible elastomeric polymer such as polyurethane, a silicone such as polydimethylsiloxane, or a film polymer such as polyethylene. An example of one such film is Entrofilm 861, available from Entroech Inc., Columbus, Ohio.

As shown in FIG. 3, an electrically insulative adhesive material 35 seals the perimeter of the layer 34 to the periphery of the PCB 21. An example of such an electrically insulative adhesive material is a polyurethane, a silicone, a polyimide, or an acrylic copolymers, either butyl or 2-ethylhexyl acrylate and acrylic acid adhesive. One example of a common adhesive used in this application is CC3-341 from Cast-Coat, Inc., West Bridgewater, Mass.

Another embodiment of the invention that comprises the one or more layers of light transmission, electrically insulative material can also include a proximity sensor, as described herein above.

iii) remote powering and controlling of the light source

The invention can further relates to a DLS that includes a light source wherein the source of electrical power and a controller for the light source are disposed remote from the DLS as opposed to having a device-borne power source, such as a battery. As illustrated in FIGS. 1A-4A, a source of power and control to the light source 20 is provided externally of DLS 11 by a system or apparatus 27 to which DLS 11 is selectively connected through electrical connector 25 for use in a medical procedure in accordance with the intended function of the invention. The system or apparatus 27 can include an image acquisition, processing and display system or apparatus that is connected during use to light source 20 by suitable power and/or control wiring 26, as described herein above. The system or apparatus 27 can also include manually operated or automatic gain control of voltage and/or current flow through the light source. The connector 25 can be a standard electrical connector, including but not limited to a TRS (tip, ring, sleeve) connector plug as illustrated. The DLS therefore includes a minimum of components and features, and results in reduced disposable costs and reduced complexity.

Non-limiting examples of a system or apparatus for powering and control of the main light source 20 and other electronic components of the device are described in U.S. Pat. No. 6,230,046, and in US Patent publication 2007-0276258, the disclosures of which are incorporated by reference in their entireties.

Alternatively, the DLS device 11 can be provided with an on-board source of power, such as a battery, and on-board circuitry and controllers, as described herein after.

An embodiment of the invention that includes the feature of a remote powering and controlling of the light source, can also include either or both of a layer or film of an electrically insulating material, and a proximity sensor, as described herein above.

iv) disposable and reusable components

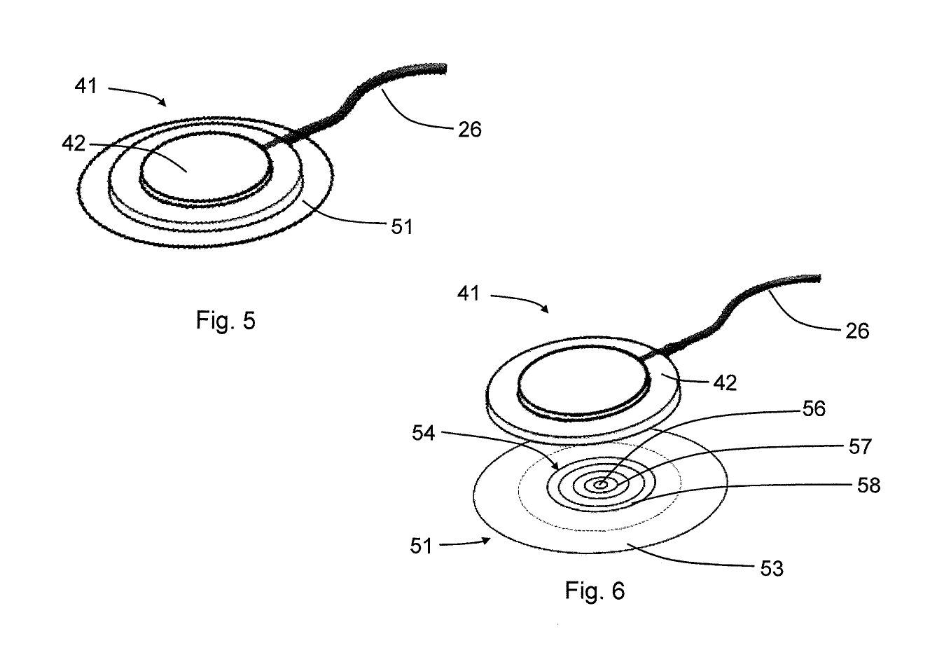

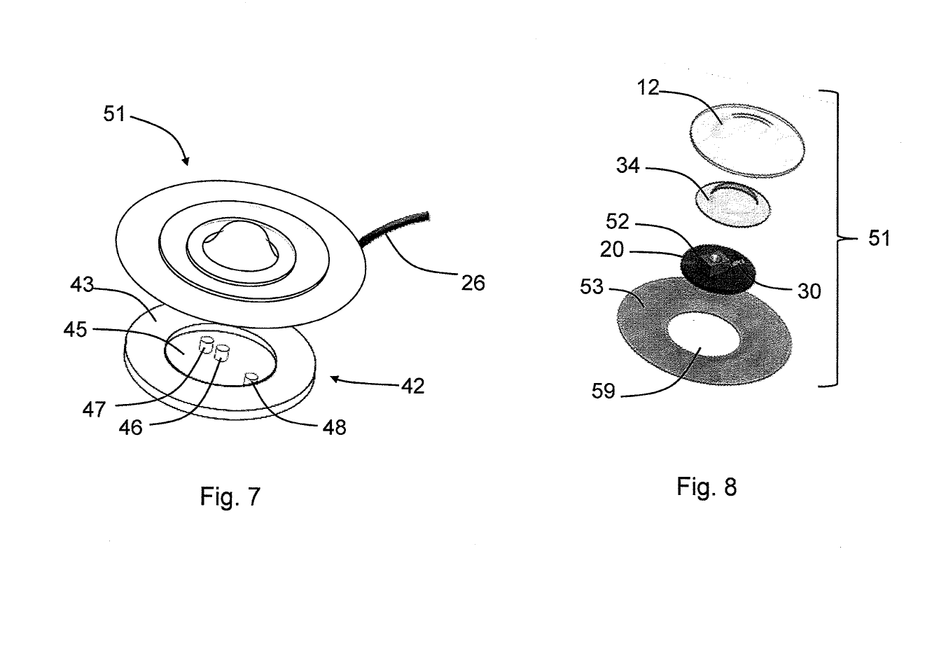

The invention further relates to a DLS that includes one or more disposable components and one or more reusable components. Referring to FIGS. 5-8, the DLS 41 includes a reusable connection portion 42 and a disposable patch portion 51. The reusable connection portion 42 illustrated in FIG. 7 includes a base 43 for supporting the light source 20, and a means for establishing electrical connection for power and control of the light source 20. Base 43 has a circular recess 45 and includes a plurality of electronic leads, illustrated as three leads 46-48. The electronic leads 46-48 are illustrated as cylindrical electrically-conductive posts having planar top surfaces, which are electronically connected to the terminal ends of wires of the power and control wiring 26 used for connecting the DLS 41 to an external source of power and controller. The electrical leads include a central post 46, an intermediate radial post 47, and a distal radial post 48. The material of the base 43 can be either a thermoplastic or thermoset polymeric material, and is preferably a material that can be sterilized for reuse after medical procedures. Examples of such materials that could be used for the base include but are not limited to polypropylene, acrylonitrile butadiene styrene (ABS), polyvinyl chloride (PVC), or even polybutylene or a suitable polyamide treated to reduce its static electrical potential.

The disposable patch portion 51 illustrated in FIGS. 6-8 includes a printed circuit board (PCB) 52 to secure the main LED 20 and any optional proximity device 30. On the opposing face 54 of the circular PCB 52 are a plurality of electronic leads, illustrated as three leads 56-58, which are connected by internal circuitry to the main LED 20 and optional proximity device 30. The illustrated three leads include a central landing lead 56, an intermediate annular landing lead 57, and a distal annular landing lead 58. Positioning of the PCB 52 of the disposable patch portion 51 into the recess 45 of the base 43 places the central, intermediate and distal posts 46,47,48 into electrical communication with the respective central, intermediate and distal landing leads 56,57,58, regardless of the radial orientation of the PCB 52 within the recess 43.

The disposable patch portion 51 also includes the conforming layer 12 covering the main LED 20, and a thin reflective layer or film 53 that extends outward from the PCB 52 and beyond the perimeter edges of the conforming layer 12. The conforming layer 12 and reflective layer 53 are of the same material as described for the earlier embodiment of DLS 11. Substantially circular aperture 59 is formed in the thin reflective layer 53 to allow the PCB 52 and its posts 46-48 to extend therethrough into position within the recess 45 of the base 43 for connection with the landing leads 56-58.

The PCB 52 of the disposable patch portion 51 may be configured to physically snap into and be restrained in the annular recess 45 of the base 43 of the reusable connection portion 42 so that the assembled unit can be used for transillumination. After use, the disposable patch portion 51, including the PCB 52 with the light source 20 and proximity sensor 30, the conforming layer 12, and the thin reflective layer 53 are removed by popping the disposable patch portion 51 off of the base 43, and discarded as a single unit. The reusable connection portion 42 can be cleaned, optionally sterilized, and reused multiple times.

An embodiment of the invention that includes the feature of a disposable component and a reusable component, can also include any one of or a combination of two, or all three of a layer or film of an electrically insulating material, a proximity sensor, and a remote powering and controlling of the light source and other electronic components, as described herein above.

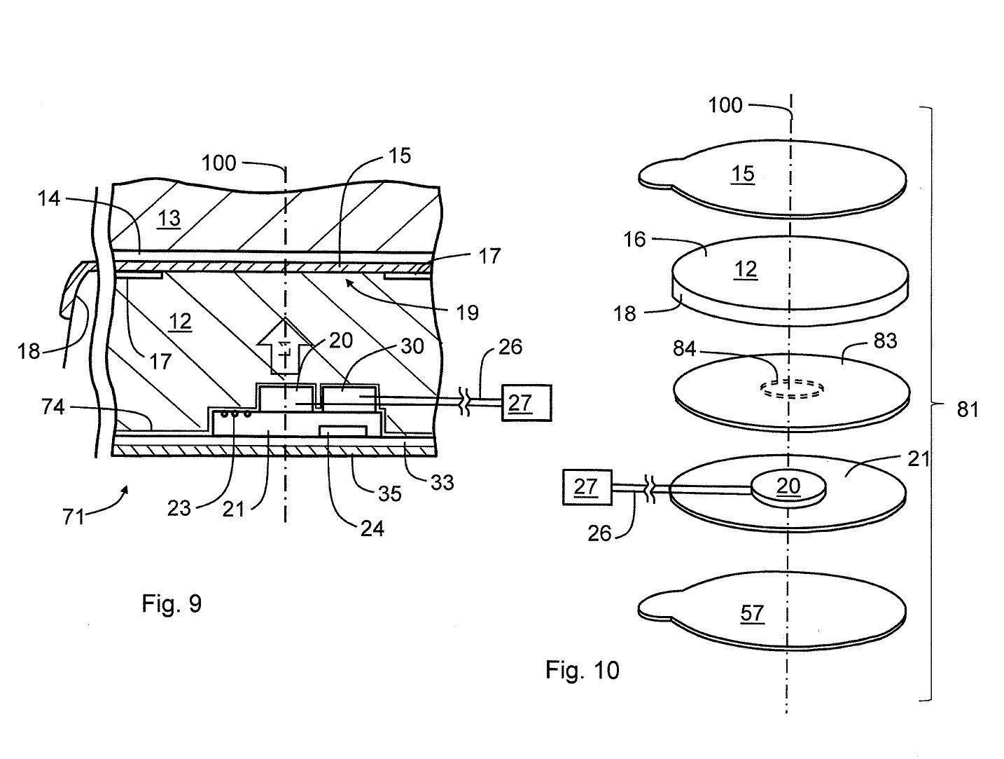

FIG. 9 shows a cross sectional view of another embodiment of a DLS 71 structure representative of the invention along with the supporting structural elements that render the structure a complete unit.

The DLS 71 includes the first substantially optically transparent conforming layer 12, as described herein above, to interface with and provide a layer conforming to the surface 14 of the body portion 13 of interest to be illuminated. A protective contaminant barrier release layer 15 of an nIR transparent polymer or other suitable material, as described herein above, covers the body-contacting surface 16 and optionally the periphery 18 of conforming layer 12 for containment and protection of conforming layer 12, and is removed and discarded prior to the application of DLS 11 to the body portion 13 surface 14 of a patient.

A light source 20 is supported on a non-conductive substrate of suitable material, such as the PCB 21 as described herein above. The light source 20 is oriented on the PCB to direct emitted light along an axis 100 of the device, substantially transverse to the body-contacting surface 16 of conforming layer 12. The PCB 21 may further support control circuitry 23 and a power source in the form of battery 24 disposed on PCB 21. For example, battery 24 may comprise a conventional coin cell battery comprising silver oxide or other material known in the art and selected by one skilled in the applicable art. The control circuitry typically consists of conducting layers, typically made of thin copper foil, and insulating layers consisting of dielectric material that are laminated together with epoxy resin. The DLS 71 also includes, mounted on the PCB 21, the proximity sensor device 30, as described herein above, that includes the light emitter LED 31 and photodetector 32, which are connected in the circuitry 23 to control activation of a light source 20 only when DLS 71 is in intimate proximity or direct contact with the surface of body portion 13 of interest.

While nIR light may be controlled electronically, it is also possible to insert semiopaque films into the structure between the nIR LED and the conforming layer shown as 12. These layers may be as specialized as a wratten photographic filter or as simple as carbon black filled polyethylene sheets that form a neutral density optical filter. In this application, a number of filters would be included in a stack so that as each one is removed the nIR light become brighter and the image produced by the nIR sensor becomes ever more useful to the clinician. This embodiment is useful in primitive applications where the availability of electrical power for a controller is unavailable or too cumbersome, e.g. a remote accident or battlefield site.

The DLS 71 also includes a layer or film of an electrically insulating material as a means for isolating electrically at least the light source from the body portion of the patient. In the illustrated embodiment shown in FIG. 9, PCB 21 and any or all of the LED light source 20, any proximity sensor 30 if used, circuitry 23, and wiring 26 if used, may be coated, typically completely coated or encased, with one or more layers 74 of a nIR-light transmissive, electrically insulative or non-conductive material, including a glass, a plastic, or other suitable material selected by the skilled artisan, to electrically insulate the light source 20, PCB 21 including its circuitry 23 and other components mounted thereon, and wiring 26 to prevent electrical current from flowing into conforming layer 12, particularly when comprising an electrically conductive material, such as hydrogel. The electrically insulative layer 74 may also provide heat insulation around PCB 21. In an embodiment of the invention employing an LED as the light source 20, a non-electrically-conductive polymer material is enveloped or coated around the entire periphery of the PCB 21, proximity sensor 30, and/or LED light source 20, prior to assembly of the DLS 71 device. Examples of such insulative enveloping material includes one of more of the following elastomeric polymeric materials such as a polyurethane, a silicone, e.g. polydimethylsiloxane, a polyester such as polyethylene terephthalate, polycarbonate or polybutyrate.

In another embodiment, the electrically insulative or non-conductive material or layer is nIR-light transmissive, and visible-light non-transmissive or opaque, An nIR transmissive, visible-light opaque layer masks or hides the DLS components (the LED, proximity sensor, etc.) without interfering with operation and imaging. An nIR transmissive, visible-light opaque layer can include a plastic film containing carbon black or blank ink, or a nIR-transmissive dye (for example, EPOLIGHT™ 1175 or 1130, or ADS900AF), An example is a polycarbonate film having IR transmission colorants, available as Makrolon 2405.

A thin reflective layer or film 33 of aluminum, aluminized or metalized polymeric film such as Mylar®, Kapton®, or Teflon® films, or other suitable material, as described herein above, may be included in the DLS 71 to constrain the emitted and reflected light L1 from main light source 20 as to not interfere with the image sensing function of the invention.

Conforming layer 12 may include an optional ring 17 of an IR opaque layer comprising carbon black loaded polymeric films, or an opaque dye (e.g., EPOLIGHT™ 1175 or 1130 or ADS900AF) in order to prevent light scattering beyond the edges of conforming layer 12. Other IR opaque layers can include black ink that uses carbon black or graphite. The ring 17 defines a central opening 19 through which the light L1 emitted from the main light source 20 passes along an axis 100 of DLS 11. The ring 17 is typically disposed proximate to the surface 16, and extends outwardly to the periphery of conforming layer 12.

The layering, coating or encasing the LED light source and other electronic components by the non-electrically-conducting material can be accomplished by various processing means, including spraying or dipping the device components with or into a molten or liquid solution of the material; sealing of a resilient film or a film structure of the material onto a substrate of the DLS structure, such as the PCB.

FIG. 10 shows an exploded view of components comprising a further embodiment and structural variation of the invention, further exemplifying the structural relationships among component parts in those further embodiments. It is seen that in the embodiments of the DLS 81 of the invention illustrated in FIG. 10, a conforming layer 12, as described herein above, is included for contacting the surface of a body portion (body portion not shown in FIG. 2) Protective contaminant barrier release layer 15, as described herein above, protects the surface 16 and periphery 18 of conforming layer 12 prior to use of DLS 81 and during the process of scanning the body surface of interest. Printed circuit board (PCB) 21 supports a light source 20 of the type as described herein above, and the printed electronic circuit 23 for the powering and controlling of the LED 20. As shown, the PCB 21 is coextensive with the footprint of the conforming layer 12, although it can also be surrounded by or enclosed within the conforming layer 12. A source of power and control 27 is selectively connected through power and control wiring 26

A reflective film 83 is disposed between the conforming layer 12 and the PCB 21, for directing back through conforming layer 12 light that might be reflected from the body surface at the interface of layer 12 with the body surface of interest (not shown in FIG. 10). A central aperture 84 in the reflective film 83 registers with the light source 20 to permit the passage of light. A thin, flexible protective layer 85 (1 to 5 mils thick, preferably 2 to 4 mils) of polyester or other suitable material can be fixed to the back surface of the PCB 21 as a protective film to any electronic circuitry thereof.

The DLS structure in any of the embodiments described herein according to the teachings of the invention may be of any convenient size consistent with the size of the person on which the DLS is used, and thicknesses of the individual layers may be selected by the skilled artisan practicing the invention within the intended scope of these teachings, the thicknesses hereinabove recited as included in demonstration of the invention being representative only. It is noted further that the invention may find use in veterinary medicine as well as for human application. The overall size of the DLS is about 0.25 to 6 inches in diameter and about 0.10 to 0.5 inch in overall thickness for use on an adult person or a neonate or pediatric patient. Minimum size of the DLS may be limited by the size of the light source selected for use in conjunction with the DLS and by the size of the body features of infant patients.

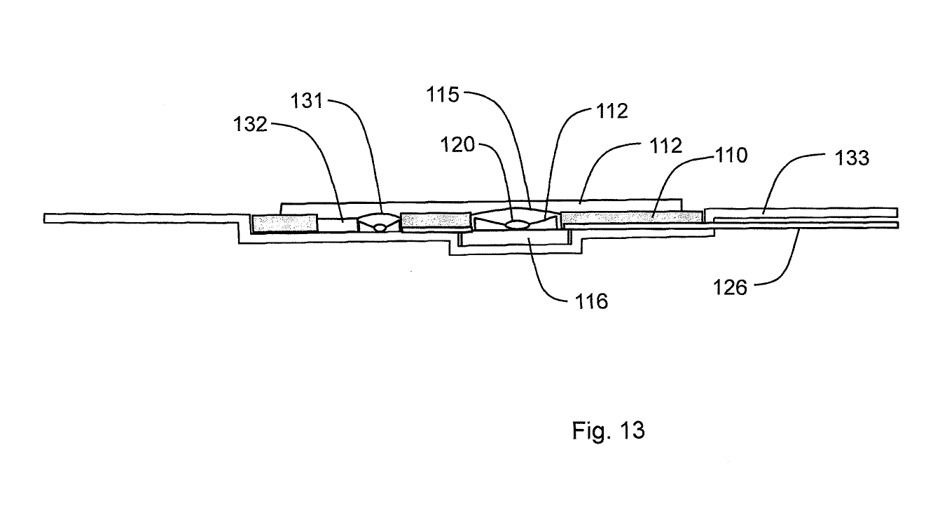

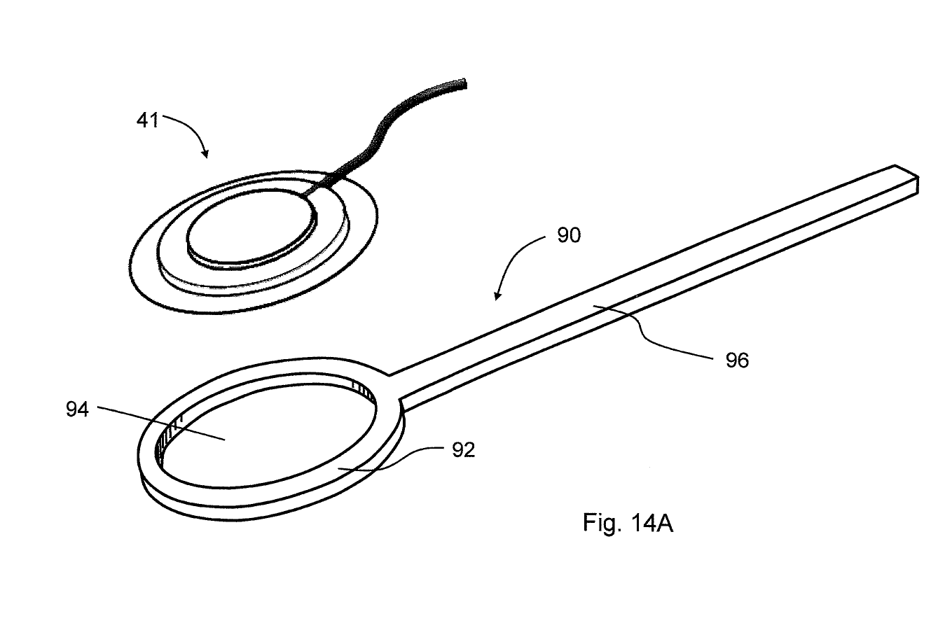

Another embodiment of a DLS is shown in FIG. 13 in cross section, including a reverse-mounted LED 120 mounted in the opening of a flexible substrate 110. The reverse mounted LED 120 has a heat sink 116 on the reverse side (facing away from the direction of light emission), with the emitted light directed and shaped with a reflector cup 114 and an optical dome 115. A proximity detector including an emitted 131 and detector 132 are mounted in flexible substrate. The LED 120, proximity emitter 131 and detector 132 are covered with a hydrogel layer 112. Circuitry 130 in the substrate 110 connects the light source and proximity detector to external wiring 126. A thin reflective film 133 overlays the substrate 110 with the reflective surface directed in the same direction as the light emission of the light source 30, with an adhesive that substantially adheres the DLS to the skin.

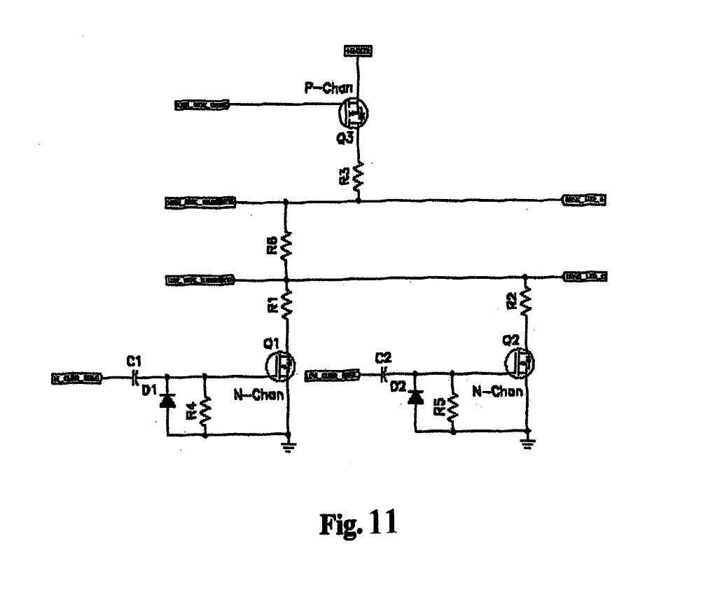

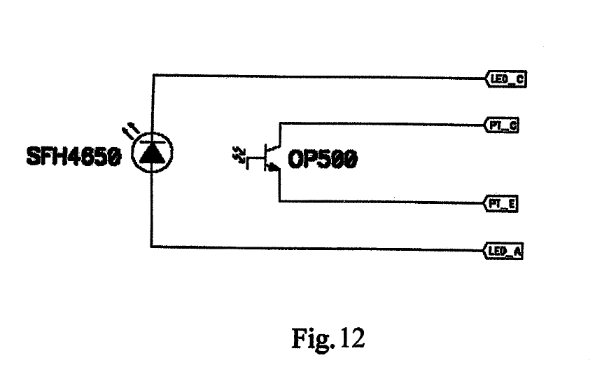

Referring now to FIG. 11, shown therein is a schematic of an exemplary circuit for DLS circuit on the printed circuit board. FIG. 12 shows a typical LED drive schematic useful in the structure of the DLS of the invention. In a representative structure of the DLS, the circuit contained on PCB 75 may include an Osram SFH 4650 MIDLED LED and an Optek OP500 series phototransistor. The onboard LED is used as the emitting device to provide proximity detection. In one (linear mode) operating mode for the DLS, the phototransistor kept from saturating by biasing with a high maximum current, several milliamps for the single phototransistor sensor. The actual current drawn is monitored and compared to programmed thresholds depending on the state of the drive for the LED (high or low power mode).

Ambient light detection is an important clue that the DLS is not properly in place. The DLS can provide a continuous background current level related to the ambient light striking the detector. If the DLS is populated with a clear lens detector, ambient light detection is improved. However, the nIR filtered version is still very sensitive to incandescent light sources.

The problem of illuminating a trauma patient in a remote setting with the necessary nIR light is made difficult because the power sources of the clinical setting are not available. For this reason the disposable light source (DLS) structure may optionally include a power source, current controller, and a proximity sensor according to the present invention as described herein.

The DLS has many functions including, 1) protecting a patient from the thermal output of the light source; 2) attaching the light source to the body to give the technician a hands free ability to attend to other matters; 3) controlling the light level administered to the patient, including the use of a series of light attenuating films that are easily removed to change the light levels; 4) activating and illuminating the light source only when the device is in position proximate to the body portion to be illuminated; 5) protecting the patient from electrical current associated with the powering or control of the light source or other components of the device; and 6) extinguishing the light source when the procedure is completed; 7) providing a barrier against the spread of infection or of disease transmission from patient to patient by virtue of it being a single use, disposable product, and (8) gently adhering to the skin surface in such a way that attachment and removal do not provide discomfort or damage to the skin, especially the sensitive skin of neonatal, pediatric and geriatric patients.

In many cases, for the DLS to function in an optimal fashion, the light source should emit as much light as possible and as necessary during its operation. One common electrical mechanism to accomplish this is to pulse the light source. In this manner the average energy requirement may be minimized, while light output maximized to the observer. Since the light source should appear to be continuous to the naked eye, the light source can be pulsed at a rate above the flicker fusion frequency of the observer, typically 60 Hz. The pulse frequency may be produced using any of a large number of electrical circuit configurations from pulsing integrated circuits (NE555) to relaxation oscillators. Further, the pulse may be linked to the imaging device so that the device is only on or has appreciable gain when the light source is emitting and is off or has very low gain when the light source is not emitting. This has the advantage of reducing the extraneous light noise seen or detected by the imaging device. This can also be utilized to eliminate any interfering pulsing signal from surrounding or ambient pulsing visible light, such as fluorescent lights, which pulse at 120 Hz (when powered from a mains circuit) and the output appears as a full wave rectified sine wave. By taking image information during the nulls in the visible ambient lighting, the noise contributed by fluorescent light, for example, to the nIR image is minimized, while the nIR image information is maximized (see e.g., U.S. Patent Publication US 2007-0276258, the entire disclosure contained therein being hereby incorporated herein by reference). The gate that admits the nIR light at the nulls in the 120 Hz cycle to the detector might be expanded to admit more or less of the ambient illumination in order to give the clinician the ability of see the back scattered light from the patients skin. This then gives the clinician both surface and subsurface images so as to direct the application of an intravenous needle into the vein.

The pulsed nIR lighting may be synchronized with a gating circuit within the image conversion detector. A linkage is provided between the nIR light source and the detector, which may be accomplished with control wiring connecting the two devices or with a signal transmitted from either the nIR light source or the image conversion detector. Either is sufficient as a trigger for the lighting and detection process. This trigger information may be provided by any convenient means as would occur to the skilled artisan practicing the invention such that the detector and light source are coordinated as to function in synchronized fashion in this embodiment of the invention. Any suitable pulsing system as would occur to the skilled artisan practicing the invention may be selected, the specific system selected not being considered limiting of the scope of the invention or the appended claims. Suitable systems contemplated for use in conjunction with the invention may include, but are not limited to temporal, spectral, amplitude, or even directional or spatial pulsing, as would occur to the skilled artisan practicing the invention.

LED emitters are particularly well suited for controlling the amount of light output as a function of the energy input of the diode. The intensity of the light emitted by the LED emitters of the DLS can be controlled, typically externally of DLS, to increase, decrease and control desired light gain and image contrast, by either manual or automated means.

For the purpose of describing the invention and defining the scope thereof, the terms “optical” or “optically” shall, in accordance with customary usage, be defined to include the ultraviolet, visible, nIR and IR regions of the electromagnetic spectrum lying between about 0.1 to about 1.4 microns.

In order to minimize reflections at the interface and to efficiently illuminate the surface of body portion with the available light from a suitable light source that is used in conjunction with the invention, the refractive index of conforming layer should be closely matched to the refractive index of the body portion of interest. The refractive index to infrared light of skin and tissue of the body is generally about 1.3 to 1.6. Accordingly, in contemplation of the invention, efficient coupling would result for a refractive index of conforming layer in the range of about 1.33 to 1.55. In order to efficiently couple light into the body portion , the conforming layer should make substantially intimate laminar contact with the body surface in order to conform to various complex surface features of the skin, such as moles or hair, or various skin conditions such as acne, psoriasis, lesions, or other blemishes that would result in pockets or gaps of air in the interface of conforming layer with the body surface that inhibit optical coupling with the body surface and result in Fresnel reflections at the interface or that result in light backscattering affecting the quality of the resulting image. Conforming layer should also provide a layer of thermal insulation between the light source (as described below) and the body surface of the skin so as to prevent discomfort to the patient or thermal damage to the skin, and to provide a sterile barrier between body surface and the light source in case the DLS or light source need to be placed near or across a wound. Materials comprising conforming layer useful in the structure of DLS as an optical coupling layer and as a thermal diffuser include polyurethane, polyethylene, glyceryl polymethacrylate, silicone particularly the gel form, polydimethylsiloxane (PDMS) polyester, polydimethylsiloxane, a hydrocolloid gel, other highly transmissive, conformable material. Other materials may be selected by one skilled in the art guided by these teachings without departing from the spirit of the invention or the scope of the appended claims, material selection therefore not considered strictly limiting of the invention herein. In the nonlimiting DLS 11 structure shown in FIG. 1, conforming layer 12 comprises a thin layer (20 to 280 mils thick, preferably 30-50 mils) (1 mil =0.001 inch) of a hydrogel such as Entrotape 974, available from Entrotech Inc, Columbus, Ohio, and Tegaderm™ (see Next-generation hydrogel films as tissue sealants and adhesion barriers, by Bennett, S. L., Melanson, D. A., Torchiana, D. F., Wiseman, D. M., Sawhney, A. S., Journal of Cardiac Surgery 2003 vol. 18, no, 6, p. 494-499 for additional types of films that could be used in this application). This material has good thermal insulative properties, good refractive index match of approximately 1.33, and conforms well to the body surface contours. In an embodiment of the invention, the hydrogel structure includes a circular area of about 2.5 cm diameter, for use on an adult person and a circular area of about 1 cm diameter for use on infant. Size (diameter or thickness) of conforming layer and overall size of DLS is not considered limiting of the invention.

The hydrogel material provides various functions, including adhesion and comfort of DLS as applied to body surface, heat shielding, transmission of light from the light source, and optical coupling of DLS to the body portion. The hydrogel material may be particularly useful in the structure of DLS in that that material can provide adhesion of DLS to the skin at its body-contacting surface, while allowing for gentle peeling and release from body surface following the procedure; which is particularly important when working with neonates, infants and elderly patients who may have fragile or pain-sensitive skin conditions or when imaging a wound in the surface of the body portion of interest.

The protective containment barrier release layer described should be light (preferably nIR light) transparent, with low scattering, and may be one, a few, or several mils thick (a mil is 1/1,000 inch). Examples of ranges of thickness of the barrier release layer are 1 to 7 mils thick, and 2 to 4 mils thick. The barrier release layer may comprise a clear, non-adhesive, and preferably nIR-transmissive, plastic film as a “release liner” that will allow the user to move DLS over the outside surface of the body portion to be imaged in order to survey the body part for the desired location for subsequent imaging. Prior to the removal of barrier release layer, DLS may be used in a scanning mode to determine the optimum location along body surface of the patient for illuminating the intended cannulation site and for placement of DLS in an imaging procedure. Once the user has selected the location for placing DLS, barrier release layer can be peeled from the body-contacting surface of conforming layer for adhesively attaching the DLS to the body portion 13 via the then-exposed surface of conforming layer.

A minimum size of DLS 11 may be limited by the size of the light source selected for use in conjunction with the DLS 11 and by the size of the body features of infant patients. The hydrogel structure defines the area or “footprint” that DLS 11 presents when applied onto the body portion 13, which can be of any convenient size consistent with the size of the person or body part of the person on which the DLS 11 is used. Typical hydrogel footprint shapes can include square, rectangular, oval and “teardrop” shapes. The invention can be used in veterinary practices on other mammals in addition to human medical practice.

The invention also includes a sterilized DLS device packaged within a suitable sterile packaging system for shipment and storage. The packaging system can include a sealed, sterile package made of air-tight and vapor-impervious material, such as aluminum foil and foil-plastic laminates. Instructions for use of the DLS device can also be provided within or on the packaging material. Thus, the DLS device can be removed from a foil pouch, electrically coupled to a detecting or visualizing apparatus, which provides power and control to the light source. After removal of the release liner to expose the hydrogel surface that contacts the skin or body portion, the DLS device is placed on a site on the patient's body. The output of the light source is typically controlled by the controller on the image acquisition or processing system. Once the vascular access procedure is complete, the DLS can be removed from the patient, unplugged from the imaging system or the power cable connected to the imaging system, and disposed of appropriately. This process can be repeated over and over with each new patient. The single-use DLS is particularly important because it eliminates the possibility that the light source can transmit infectious organisms between patients.

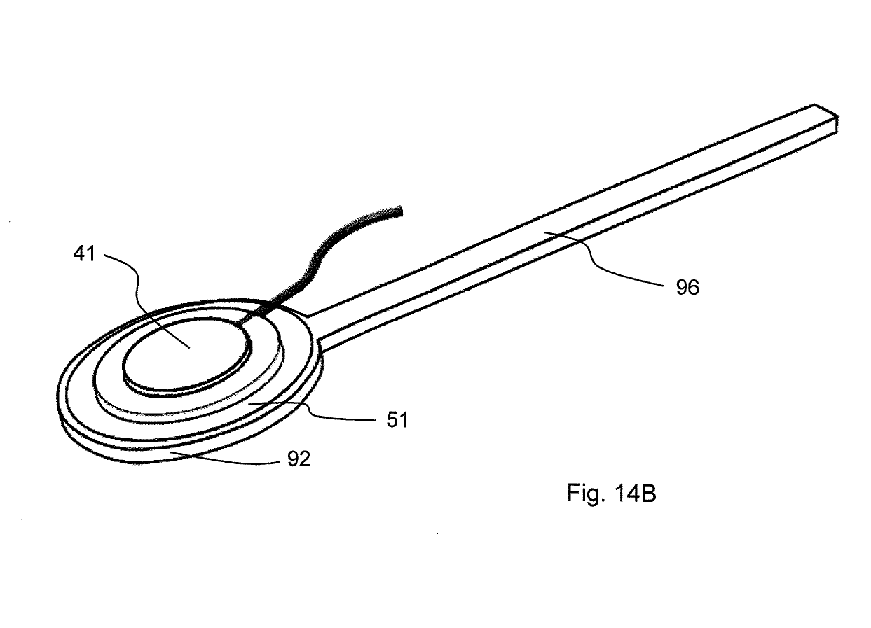

FIGS. 14A and 14B shows an alternative use for the DLS as a manipulable and mobile light source during medical procedures. The DLS 41 can be attached to an implement, illustrated as a mounting handle 90 for manually positioning the DLS near or against the skin of the patient, or near, on or within the body of the patient. The adhesive film on the underside of the outer annular portion 53 of the DLS can be adhered to the similarly shaped and sized frame 92 of the handle 90, permitting the emitted light of the DLS to pass through the opening 94 in the frame. The mounting handle 90 can be made of plastic, wood, any metal, or other material, or a composite, and the mount frame and/or the handle can be rigid, flexible, or malleable to shape the handle as needed for a particular procedure. The implement can be a sterile article, and can be reusable or disposable.

It is understood that modifications to the invention may be made as might occur to one with skill in the field of the invention within the scope of the appended claims. All embodiments contemplated hereunder that achieve the objects of the invention have therefore not been shown in complete detail. Other embodiments may be developed without departing from the spirit of the invention or from the scope of the appended claims.

1. A disposable light source (DLS) device for use in medical imaging procedures in the visualization of subcutaneous structures of a body portion, comprising,
an optically transparent conforming layer having a first surface configured for interfacing in intimate laminar contact with the surface of a body portion of interest to adhere the DLS device to the body portion and for providing optical coupling with the body portion, the conforming layer having a second surface opposite the first surface; and
a first light source for emitting light through the conforming layer and illuminating the body portion of interest, and further including electrical circuitry adapted for selectively activating the light source whereby the body portion of interest is selectively illuminated through the conforming layer.

2. The device according to claim 1, further including at least one light transmissive and electrically insulative layer that is disposed between and electrically insulates the first light source from the conforming layer.

3. The device according to claim 1, wherein the first light source emits light in the near infrared.

4. The device according to claim 3 wherein the first light source emits light in a first wavelength within the spectral range from about 0.7 to 1.4 microns.

5. The device according to claim 1 wherein the first light source is pulsed.

6. The device according to claim 1 wherein the first light source is supported on a printed circuit board, and the printed circuit board and its connectors are encased in the at least one light transmissive and electrically insulative layer.

7. The device according to claim 6 wherein the printed circuit board further supports a source of power disposed thereon and operatively attached to the light source.

8. The device according to claim 1 further comprising a film having a reflective surface oriented toward the conforming layer, the film selected from the group consisting of aluminum and an aluminized or metalized polymeric film.

9. The device according to claim 8 wherein the reflective surface confronts the second surface of the conforming layer and has a central opening in registration with the first light source.

10. The device according to claim 1 further including a non-adhesive, removable, optically transparent film disposed on the first surface of the conforming layer.

11. The device according to claim 2 wherein the first light source is a light emitting diode (LED).

12. The device according to claim 11 wherein the LED is a reverse-mounted LED.

13. The device according to claim 11, further comprising a proximity sensor that controls activation of the first light source only when the conforming layer is brought into proximity to the body surface.

14. The device according to claim 13 wherein the proximity sensor includes a second light source and a photodetector for detecting reflected light from the second light source.

15. The device according to claim 14 wherein the second light source emits light at a second wavelength in the range of about 0.890 microns to 1.00 microns.

16. The device according to claim 13 wherein the proximity sensor includes a photodetector for detecting reflected light from the first light source.

17. The device according to claim 16 wherein the first light source emits light that includes a distinct light emission pattern, the photodetector produces a detection signal from the reflected light from the first light source, and a controller analyzes the detection signal for the distinct light emission pattern.

18. The device according to claim 16 wherein device operates in a first proximity mode wherein the first light source emits light at a low-power emitting state, and includes a low-power threshold wherein an amount of reflected light from the first light source detected by the photodetector that exceeds the low-power threshold, the device operates in a second imaging mode wherein the first light source emits light at a high-power emitting state, and includes a high-power threshold wherein when an amount of reflected light from the first light source detected by the photodetector in the high-power emitting state is less than the high-power threshold, the device operates in the first proximity mode.

19. The device according to claim 11, wherein the device does not include a source of electrical power or an electronic controller for powering and control of the first light source; but does include two or more electrical terminals connected to the first light source for electrically connecting the first light source to an external, remote power and control apparatus.

20. (canceled)

21. The device according to claim 11, wherein the intimate laminar contact of the conforming layer with the surface of the body portion provides a self-adhering, hands-free attachment, sufficient to allow the DLS device to remain in position on the body portion during the imaging procedure, without requiring holding or adjustment by hand of the operator.
